(19) United States
(12) Patent Application Publication
(10) Pub. No.: US 2003/0054246 A1
(43) Pub. Date: Mar. 20, 2003

(54) Zinc anode for electrochemical cells

(76) Inventors: Lin-Feng Li, Croton, NY (US); Dan K. Nguyen, Bronx, NY (US)

Correspondence Address: Ralph J. Crispino, Reveo, Inc., 85 Executive Blvd., Elmsford, NY, 10523, US

(21) Appl. No.: 10/174,219

(22) Filed: Jun. 17, 2002

Related U.S. Application Data
(60) Provisional application No. 60/298,537, filed on Jun. 15, 2001.

(51) Int. Cl. H01M 4/62; C 07D 3 7/12

(52) U.S. Cl. 429/214; 429/229; 549/429; 549/497

(57) ABSTRACT

An additive compound for oxidizable metal such as zinc is provided. The additive includes a sorbitan based compound of the formula:

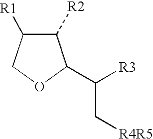

wherein R1, R2, R3 may be the same or different, and are each selected from the group consisting of OH and (OCX₁X₂CX₃X₄)ₙOH, where X₁ X₂, X₃, X₄ are selected from the group consisting of H, F, and an aliphatic group, wherein n is between 1 and about 10000;

R4 is selected from the group consisting of a single bond, OH and (OCX₁X₂CX₃X₄)ₙOH, where X₁ X₂, X₃, X₄ are selected from the group consisting of H, F, and an aliphatic group, wherein n is between 1 and about 10000; and

R5 is selected from the group consisting of OR6 and OOCR6, wherein R6 is an aliphatic group.

### RELATED APPLICATIONS

The present application claims priority to Unites States Provisional Patent Application Ser. No. 60/298,537 entitled “ZINC ANODE FOR ELECTROCHEMICAL CELLS” filed on Jun. 15, 2001 by the inventors herein, the entire disclosure of which is incorporated herein by reference in its entirety.

### BACKGROUND OF THE INVENTION

1. Field Of The Invention

This invention relates to zinc anode for electrochemical cells, and particularly to corrosion inhibiting anode materials.

2. Description Of The Prior Art

Zinc and zinc alloys have been used for many years as active materials in electrochemical cells, including zinc air, zinc-silver, zinc-manganese, zinc nickel, zinc halide, and other cell systems. Zinc is preferred in many applications because of the relatively high energy densities, as well as its natural abundance. However, one associated problem with zinc-based electrochemical cells relates to self discharge, generally wherein hydrogen gasses from the system upon self -discharge of the cell. This effect, generally referred to as anode corrosion in many cell systems, detriments the life of the cell, as zinc is consumed for the production of unwanted hydrogen gas rather than energy. Further, such gas evolution will increase the cell internal pressure, which may lead to leakage of the electrolyte.

Previous attempts at suppressing possible corrosion of the zinc powder as an anode active material employed mercury-containing zinc alloys. Certain mercury-containing zinc effectively maintained an acceptable storing property of a cell having such an anode. However, due to environmental concerns, decreasing mercury content in the anode zinc alloy powder and commercialization of a battery including a non-amalgamated, mercury free zinc alloy powder have been demanded in recent years.

Typical approaches included incorporating any of bismuth, aluminum, calcium, indium, tin, and other materials to impart corrosion-resistant properties and suppress the generation of gas due to the corrosion of the zinc powder. This zinc alloy powder is thus taken as a promising anode zinc material for the mercury free alkaline battery. However, oftentimes, these various alloy components still result in a significant amount of hydrogen evolution during the extended storage of the cell.

Other approaches to varying anode material by surface treatment of the anode with the certain polymers or surface active agents, or alternatively electrolytes, have been attempted, however, most generally result in similar outcomes. That is, while corrosion reduction has been possible, heretofore the discharge capacity of such corrosion systems is lacking.

Accordingly, it would be desirable to provide an anti-corrosion zinc anode material while maintaining suitable discharge capacities, particularly at high discharging rate.

### SUMMARY OF THE INVENTION

The above-discussed and other problems and deficiencies of the prior art are overcome or alleviated by the several compositions, wherein a zinc material is coated with an inhibitor compound. The inhibitor compound generally comprises a surfactant having the general formula:

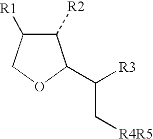

wherein R1, R2, R3 may be the same or different, and are each selected from the group consisting of OH and (OCX₁X₂CX₃X₄)ₙOH, where X₁ X₂, X₃, X₄ are selected from the group consisting of H, F, and an aliphatic group such as CH₃ or CH₃CH₂) (e.g., (OCH₂CH₂)ₙOH), wherein n is between 1 and about 10000;

R4 is selected from the group consisting of a single bond, OH and (OCX₁X₂CX₃X₄)ₙOH, where X₁ X₂, X₃, X₄ are selected from the group consisting of H, F, and an aliphatic group such as CH₃ or CH₃CH₂) (e.g., (OCH₂CH₂)ₙOH), wherein n is between 1 and about 10000; and

R5 is selected from the group consisting of OR6 and OOCR6, wherein R6 is an aliphatic group.

The above-discussed and other features and advantages of the present invention will be appreciated and understood by those skilled in the art from the following detailed description and drawings.

### BRIEF DESCRIPTION OF THE DRAWINGS

FIG. 1 is a representation of discharge capacity data for various electrochemical cells using different anode materials including a zinc material having an inhibitor compound therein as described herein.

### DETAILED DESCRIPTION OF THE ILLUSTRATIVE EMBODIMENTS

An inhibitor compound for use with a zinc based electrode material generally comprises a sorbitan based compound of the general formula:

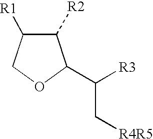

In formula (1), R1, R2, R3 may be the same or different, and are each selected from the group consisting of OH and (OCX₁X₂CX₃X₄)ₙOH, where X₁ X₂, X₃, X₄ are selected from the group consisting of H, F, and an aliphatic group such as CH₃ or CH₃CH₂) (e.g., (OCH₂CH₂)ₙOH), wherein the values for n (hereinafter n_{R1}, n_{R2}, n_{R3}) are generally each between 1 and about 10000. R4 is selected from the group consisting of a single bond, OH and (OCX₁X₂CX₃X₄)ₙOH, where X₁ X₂, X₃, X₄ are selected from the group consisting of H, F, and an aliphatic group such as CH₃ or CH₃CH₂) (e.g., (OCH₂CH₂)ₙOH), wherein n (hereinafter n_{R4}) is between 1 and about 10000. In a preferred embodiment, the SUM Of n_{R1}, n_{R2}, n_{R3,} and n_{R4} is less than about 10000, more preferably less than about 1000, and most preferably less than about 200. Further, R5 is generally an ester compound or an ether compound, selected from the group consisting of −OR6 and −OOCR6, wherein R6 is an aliphatic group.

Examples of compounds of the general formula (1) useful as inhibitor compounds include, but are not limited to: polyoxyethylene sorbitanaliphatic acid esters such as polyoxyethylene sorbitan monolaurate, polyoxyethylene sorbitan monopalmitate, polyoxyethylene sorbitan monostearate, polyoxyethylene sorbitan monooleate, polyoxyethylene sorbitan trioleate, and polyoxyethylene sorbitan tristearate; and: sorbitanaliphatic acid esters such as sorbitan monolaurate, sorbitan monopalmitate, sorbitan monostearate, sorbitan monooleate, sorbitan trioleate, and sorbitan tristearate. However, one of skill in the art will appreciate that other sorbitan based compounds of the general formula (1) may be employed. Further, various combinations of inhibiting compounds of the formula (1) may be employed.

The inhibitor compound may be incorporated in a zinc or zinc alloy anode material or an electrochemical cell based on such zinc anode material in several ways. For example, the inhibitor compound may be added to an electrolyte, which may be gelled and mixed with the zinc material. Alternatively, the inhibitor compound can be added directly to the zinc anode material. For example, the inhibitor compound may be dissolved in a suitable solvent such as acetone or water to form a diluted solution. The zinc material may be soaked in the solution, whereby the solvent is subsequently evaporated at room temperature or an elevated temperature such as 50° C. Alternatively, the diluted inhibitor compound solution may be sprayed on the zinc material. Further, the zinc material may be tumbled with a solution of the inhibitor compound and subsequently dried. Regardless of the method employed, the inhibitor compound is preferably distributed homogeneously throughout the zinc material to provide uniform corrosion resistance.

The inhibitor compound is provided in an amount that is sufficient to inhibit or substantially prevent the occurrence of anode corrosion. In general, amounts of about 1 part per million (ppm) to about 5% may be employed, preferably about 50 ppm to about 2000 ppm, more preferably about 50 ppm to about 200 ppm with respect to the weight of zinc material. It will be appreciated from one skilled in the art that the actual amount of the inhibitor compound necessary may be determined by well-known methods.

The zinc material may comprise zinc metal. Zinc may also be alloyed with constituents including, but not limited to, bismuth, calcium, magnesium, aluminum, lithium, indium, lead, mercury, gallium, tin, cadmium, germanium, antimony, selenium, thallium, or combinations comprising at least one of the foregoing constituents. The metal constituent may be provided in the form of foil, powder, dust, granules, flakes, needles, pellets, fibers, or other particles.

The zinc material coated with the inhibitor compound may be employed as a dry material, i.e., without electrolyte. Such material is useful, for example, in electrochemical cell systems where the electrolyte is provided independent of the zinc material.

Alternatively, the zinc material may be incorporated with an electrolyte, for example, to form a zinc paste. Suitable electrolyte materials include ion conducting material to allow ionic conduction between the metal anode and the cathode. An ion conducting amount of electrolyte may be provided in the anode material. The electrolyte generally comprises ionic conducting materials such as KOH, NaOH, LiOH, other materials, or a combination comprising at least one of the foregoing electrolyte media. Particularly, the electrolyte may comprise aqueous electrolytes having a concentration of about 5% ionic conducting materials to about 55% ionic conducting materials, preferably about 10% ionic conducting materials to about 55% ionic conducting materials, and more preferably about 35% ionic conducting materials to about 45% ionic conducting materials.

A gelling agent may also be used in sufficient quantity to provide the desired consistency of the paste. The percentage of gelling agent (based on the total electrolyte without zinc material) is generally about 0.2% to about 20%, preferably about 1% to about 10%, more preferably about 1% to about 5%. The gelling agent may be a crosslinked polyacrylic acid (PAA), such as the Carbopol® family of crosslinked polyacrylic acids (e.g., Carbopol® 675, Carbopol® 940) available from Goodrich Corp., Charlotte, N.C., and potassium and sodium salts of polyacrylic acid or polymethyl acrylic acid; carboxymethyl cellulose sodium salt (CMC), such as those available from Aldrich Chemical Co., Inc., Milwaukee, Wis.; hydroxypropylmethyl cellulose; polyvinyl alcohol (PVA); poly(ethylene oxide) (PEO); polybutylvinyl alcohol (PBVA); natural gum; Polygel 4P (available from Sigma-Aldrich); grafted starch, such as Waterlock® A221, available from Grain Processing Corp., Muscatine, Iowa.; combinations comprising at least one of the foregoing gelling agents; and the like.

Further, the zinc paste may include zinc oxide, generally to provide further reduction in gassing of the material. The zinc oxide may be included in the electrolyte or in the zinc or zinc alloy material.

The invention will now be described by way of a non-limiting example.

### Example

A zinc material having an embodiment of the inhibitor compound was prepared as follows.

```
A zinc alloy was provided having the following assay:
102 ppm Bi
200 ppm In
  99 ppm Al
  33 ppm Pb (unavoidable impurity)
  2 ppm Fe (unavoidable impurity)
  5 ppm Cu (unavoidable impurity)
  1 ppm Cd (unavoidable impurity)
```

The zinc alloy further had a particle distribution as follows:

| | |
|---|---|
| +425 micrometers | 0% |
| +250 micrometers | 23% |
| +150 micrometers | 37% |
| +106 micrometers | 24% |
| +75 micrometers | 15% |
| −75 micrometers | 2% |

The zinc alloy was tumbled with a 1% solution of an inhibitor compound comprising polyoxyethylene(20) sorbitan monooleate in water. The tumbled mixture was dried at 50° C. until substantially all of the solvent evaporated. Overall, about 0.01 wt. %, based on the weight of the zinc alloy, of the inhibitor compound (without solvent) was provided to coat the zinc alloy.

Gassing tests were performed with 5 grams the zinc material incorporating the inhibitor compound with 10 milliliters of a 40% KOH saturated with zinc oxide solution. The test demonstrated that the anode material incorporating the inhibitor compound did not gas after one month at 50° C. Further, at 70° C., gassing tests demonstrated that the anode material incorporating the inhibitor compound evolved less than 3 to about 4 microliters per day for seven days.

To verify discharging capacities of electrochemical cells formed with the anode material incorporating the inhibitor compound, a cell was constructed employing the anode material in combination with an electrolyte material. The electrolyte material comprised about 1.8% Carbopol 675 (based on the total amount of the electrolyte material) in a 45% KOH aqueous solution. Additionally, 4% zinc oxide was incorporated into the electrolyte material.

The positive electrode used for each of the cells (using the zinc anode material and using comparative anode materials) of a test cell comprised an air diffusion cathode having a cobalt tetramethoxyphenylporphyrin (CoTMPP) catalyst supported on carbon with Teflon® binder. The air diffusion cathode was optimized for 100 mA/cm² discharge current density. Nickel sponge current collectors were used in electrical contact with the cathodes, and 2 millimeter thick copper foil current collectors were used for the anode.

Comparative cells were formed from anode material obtained from existing D-sized batteries. These batteries included Energizer® (consumer), Energizer® (industrial), Energizer® Titanium e², Duracell®, and Rayovac® Maximum™.

Two cell were assembled using a 2 millimeter thick layer of anode paste applied to a 3 cm by 5 cm cathode and separator (Freudenberg FS2213E non woven nylon separator). A cell was constructed using the above described anode paste comprising polyoxyethylene(20) sorbitan monooleate as an inhibitor compound. Likewise, anode material from the existing batteries was also used to form the comparative cells.

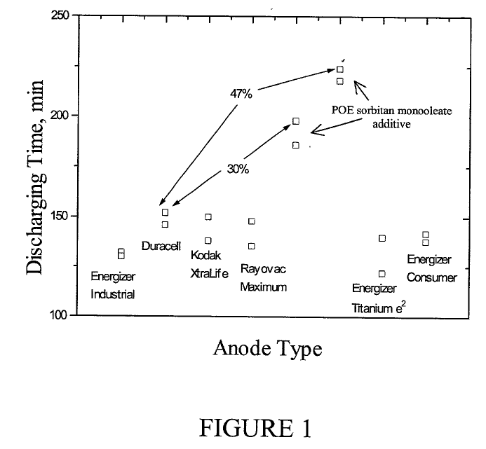

The cells were all discharged at 1.5 Amps constant current. In general, the cells formed from the anode material described herein outperformed the cells formed from the anode material from the existing batteries. FIG. 1 provides the discharging times for the various cells (two tests per cell in the example).

As can be seen from the FIG. 1, the discharge capacity of the present anode material including the inhibitor compound is increased by about 30% to up to about 45% as compared to anode material from existing cells. This benefit was achieved while minimizing corrosion and gassing of the cell due to unwanted reaction of the anode material.

Various benefits may be derived from the zinc material and electrochemical cells using the zinc material. Particularly, the anode material minimizes or eliminates self discharge and other detriments associated with gassing of zinc material. Further, the discharging capacities of the anode materials described herein are improved as compared to existing anode materials. Further, the inhibitor compound may also be used as additives for other oxidizable metal anodes.

While preferred embodiments have been shown and described, various modifications and substitutions may be made thereto without departing from the spirit and scope of the invention. Accordingly, it is to be understood that the present invention has been described by way of illustrations and not limitation.

What is claimed is:

1. An additive compound for oxidizable metals comprising a sorbitan based compound of the formula: 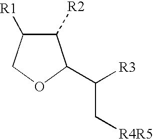
wherein R1, R2, R3 may be the same or different, and are each selected from the group consisting of OH and (OCX₁X₂CX₃X₄)ₙOH, where X₁ X₂, X₃, X₄ are selected from the group consisting of H, F, and an aliphatic group, wherein n is between 1 and about 10000;
R4 is selected from the group consisting of a single bond, OH and (OCX₁X₂CX₃X₄)ₙOH, where X₁ X₂, X₃, X₄ are selected from the group consisting of H, F, and an aliphatic group, wherein n is between 1 and about 10000; and
R5 is selected from the group consisting of OR6 and OOCR6, wherein R6 is an aliphatic group.

2. A zinc anode material comprising zinc and the additive compound of claim 1.

3. The zinc anode material as in claim 2 wherein additive compound comprises about 1 ppm to about 5%, on a weight basis, with respect to the

4. The zinc anode material as in claim 2 wherein additive compound comprises about 50 ppm to about 2000 ppm, on a weight basis, with respect to the zinc.

5. The zinc anode material as in claim 2 wherein additive compound comprises about 50 ppm to about 200 ppm, on a weight basis, with respect to the zinc.

6. The zinc anode material as in claim 2, wherein compound (1) is selected from the group consisting of polyoxyethylene sorbitanaliphatic acid esters such as polyoxyethylene sorbitan monolaurate, polyoxyethylene sorbitan monopalmitate, polyoxyethylene sorbitan monostearate, polyoxyethylene sorbitan monooleate, polyoxyethylene sorbitan trioleate, and polyoxyethylene sorbitan tristearate.

7. The zinc anode material as in claim 2, further comprising an alloy constituents selected from the group consisting of bismuth, calcium, magnesium, aluminum, lithium, indium, lead, mercury, gallium, tin, cadmium, germanium, antimony, selenium, thallium, and a combination comprising at least one of the foregoing alloy constituents.

8. The zinc anode material as in claim 2, further comprising an electrolyte.

9. The zinc anode material as in claim 8, wherein the electrolyte comprises ionic conducting material selected from the group consisting of KOH, NaOH, LiOH, and a combination comprising at least one of the foregoing ionic conducting materials.

10. The zinc anode material as in claim 8, further comprising a gelling agent.

11. In an electrochemical cell having a zinc anode and a cathode in substantially electrical isolation and in ionic communication, the improvement comprising an amount of the inhibitor compound of claim 1 in said zinc anode.
